(19) United States
(12) Patent Application Publication
(10) Pub. No.: US 2025/0064300 A1
(43) Pub. Date: Feb. 27, 2025

(54) FLUIDIC PATHS FOR AN ENDOSCOPE VALVE

(71) Applicant: Boston Scientific Scimed, Inc., Maple Grove, MN (US)

(72) Inventors: Rosangel Aimee Ramos Espinoza, Allston, MA (US); Brian Luis, Boylston, MA (US)

(73) Assignee: Boston Scientific Scimed, Inc., Maple Grove, MN (US)

(21) Appl. No.: 18/810,159

(22) Filed: Aug. 20, 2024

Related U.S. Application Data
(60) Provisional application No. 63/520,808, filed on Aug. 21, 2023.

(51) Int. Cl. A61B 1/00

(52) U.S. Cl. CPC A61B 1/00068

(57) ABSTRACT

Devices, systems, and methods for an air and water valve assembly for a medical device. The valve assembly includes a valve cap and valve stem with a central lumen running from an air inlet in the side wall of the valve stem to an air hole the valve cap. Improvements to the valve include adding an air outlet lumen, adding a seal to the air outlet lumen, and/or adding directional ribbing to a seal for water flow.

### CROSS-REFERENCE TO RELATED APPLICATIONS

This application claims the benefit of U.S. Provisional Patent Application Ser. No. 63/520,808 filed on Aug. 21, 2023, the disclosure of which is incorporated herein by reference.

### TECHNICAL FIELD

This disclosure relates generally to valve assemblies and methods, and particularly to air and water supply valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a valve assembly for a medical device, comprising a valve body having an air inlet passage, an air outlet passage, a water inlet passage, and a water outlet passage; a valve cap having an air hole; and a valve stem connected to the valve cap and configured to translate within the valve body between an upper position and a lower position, the valve stem comprising: an air inlet, a central lumen extending from the air inlet in the valve stem to the air hole in the valve cap, and an air outlet lumen extending from the central lumen to a side wall of the valve stem, the air outlet lumen configured to fluidly communicate with the air outlet passage of the valve body when the valve stem is in the upper position.

Alternatively or additionally, the valve can further comprise an air outlet seal in the air outlet lumen, the seal configured to extend into the air outlet passage of the valve body when the valve stem is in the upper position and to move with the air outlet lumen without obstructing movement of the valve stem when the valve stem translates from the upper position to the lower position. The air outlet seal can be a wiper seal.

Alternatively or additionally to any of the examples above, the air outlet seal is a flexible seal formed of a material with a durometer less than the durometer of a material forming the valve stem.

Alternatively or additionally to any of the examples above, the valve can further include an upper seal extending circumferentially around the valve stem at an axial position above the air outlet lumen and below the valve cap, the upper seal having a wiper flange contacting the valve body when the valve stem is positioned within the valve body.

Alternatively or additionally to any of the examples above, the valve can further include a middle seal extending circumferentially around the valve stem at an axial position below the air outlet lumen and above the air inlet, the middle seal having a wiper flange contacting the valve body when the valve stem is in the lower position within the valve body.

Alternatively or additionally to any of the examples above, the valve can further include a lower seal extending circumferentially around the valve stem at an axial position below the air inlet, the lower seal having first and second wiper flanges positioned such that when the valve stem is in the upper position within the valve body, the second wiper flange is above the water inlet passage of the valve body and contacts the valve body to obstruct water flow between the water inlet passage and the water outlet passage and when the valve stem is in the lower position within the valve body, the first wiper flange is above the water inlet and outlet passages of the valve body and contacts the valve body to obstruct water flow from moving within the valve body above the lower seal, while the second wiper flange is below the water inlet and outlet passages of the valve body to permit water flow between the water inlet passage and the water outlet passage.

Alternatively or additionally to any of the examples above, at least one seal around the valve stem can be a flexible seal formed of a material with a durometer less than the durometer of a material forming the valve stem.

In another example, a valve assembly for a medical device, can include a valve body having an air inlet passage, an air outlet passage, a water inlet passage, and a water outlet passage; a valve cap having an air hole; and a valve stem connected to the valve cap and configured to translate within the valve body between an upper position and a lower position. The valve stem can include an air inlet, a central lumen extending from the air inlet in the valve stem to the air hole in the valve cap, and a lower seal extending circumferentially around the valve stem at an axial position below the air inlet. The lower seal can include a first wiper flange, a second wiper flange below the first wiper flange, and a seal body between the first and second wiper flanges comprising a ribbed surface configured to direct water flowing past the seal body in a preferred rotational direction. The lower seal can be positioned on the valve stem such that when the valve stem is in the upper position within the valve body, the second wiper flange is above the water inlet passage of the valve body and contacts the valve body to obstruct water flow between the water inlet passage and the water outlet passage, and when the valve stem is in the lower position within the valve body, the first wiper flange is above the water inlet and outlet passages of the valve body and contacts the valve body to obstruct water flow from moving within the valve body above the lower seal, while the second wiper flange is below the water inlet and outlet passages of the valve body to permit water flow between the water inlet passage and the water outlet passage.

The valve assembly of claim 9, the valve stem further comprising an exterior recess such that, when the valve stem is in the upper position within the valve body, a passage is formed between the stem and the valve body to permit air flow between the air inlet passage and the air outlet passage.

Alternatively or additionally to any of the examples above, the valve stem can further include an air outlet lumen extending from the central lumen to a side wall of the valve stem, the air outlet lumen configured to fluidly communicate with the air outlet passage of the valve body when the valve stem is in the upper position.

Alternatively or additionally to any of the examples above, the valve assembly can further include an air outlet seal in the air outlet lumen, the seal configured to extend into the air outlet passage of the valve body when the valve stem is in the upper position and to move with the air outlet lumen without obstructing movement of the valve stem when the valve stem translates from the upper position to the lower position.

Alternatively or additionally to any of the examples above, the valve assembly can further include an upper seal extending circumferentially around the valve stem, the upper seal having a wiper flange contacting the valve body at an axial position below the valve cap and above the air outlet passage of the valve body when the valve stem is in the upper position within the valve body.

Alternatively or additionally to any of the examples above, the valve assembly can further include a middle seal extending circumferentially around the valve stem at an axial position above the air inlet, the middle seal having a wiper flange contacting the valve body when the valve stem is in the lower position within the valve body.

Alternatively or additionally to any of the examples above, at least one seal around the valve stem can be a flexible seal formed of a material with a durometer less than the durometer of a material forming the valve stem.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a perspective view of an illustrative supply valve;

FIG. 3A depicts a schematic cross-section view of an illustrative supply valve, with the valve in a first configuration;

FIG. 3B depicts a schematic cross-section view of an illustrative supply valve, with the valve in a second configuration;

FIG. 3C depicts a schematic cross-section view of an illustrative supply valve, with the valve in a third configuration;

FIG. 4 depicts a schematic cross-section view of an illustrative valve stem with an air outlet lumen.

FIG. 5A depicts a schematic cross-section view of an illustrative valve stem with an air outlet lumen and air outlet seal.

FIGS. 5B and 5C depict exterior side views of an illustrative valve stem with an air outlet seal.

FIG. 5D depicts an above perspective view of an illustrative valve cap with an indicator for an air outlet lumen and air outlet seal.

FIG. 6 depicts a schematic cross-section view of an illustrative valve stem with an expanded exterior recess.

FIG. 7 depicts an exterior side view of an illustrative valve stem with a lower seal having a ribbed surface.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

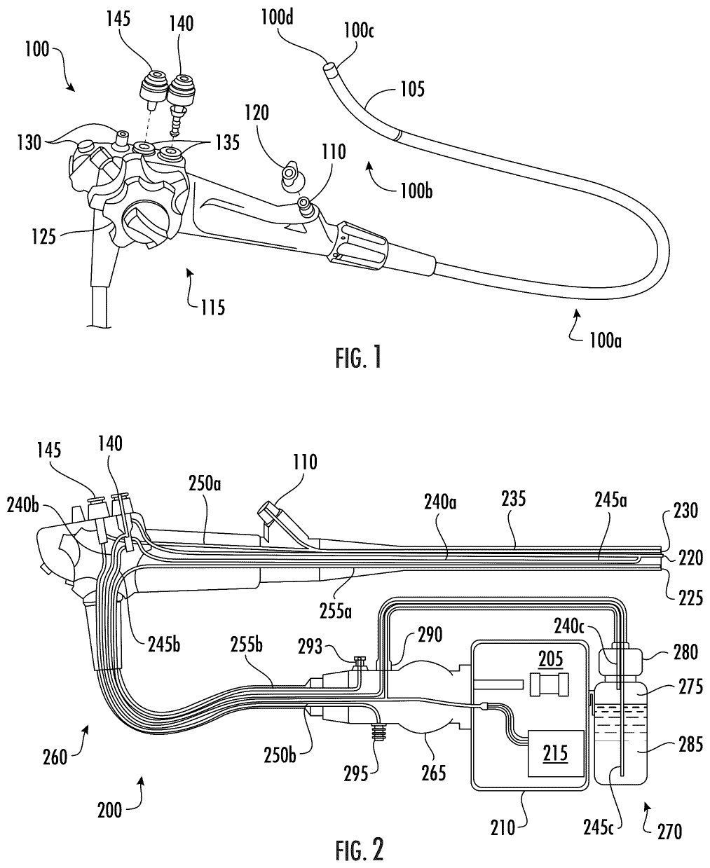

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

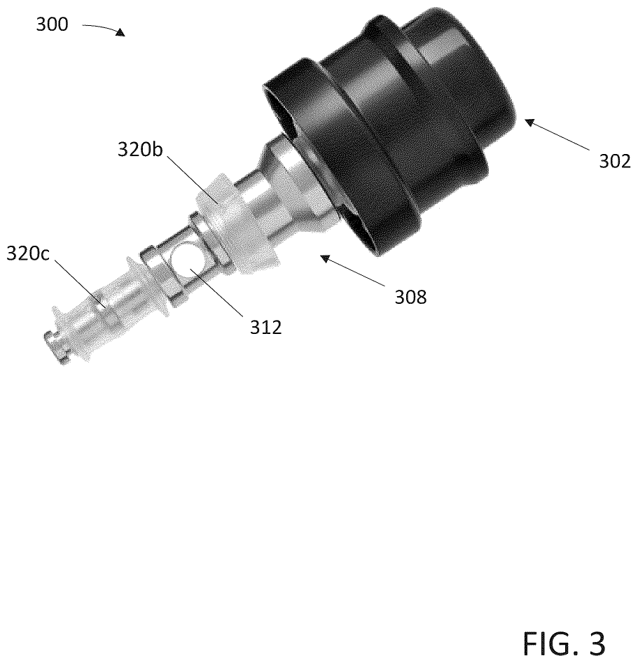

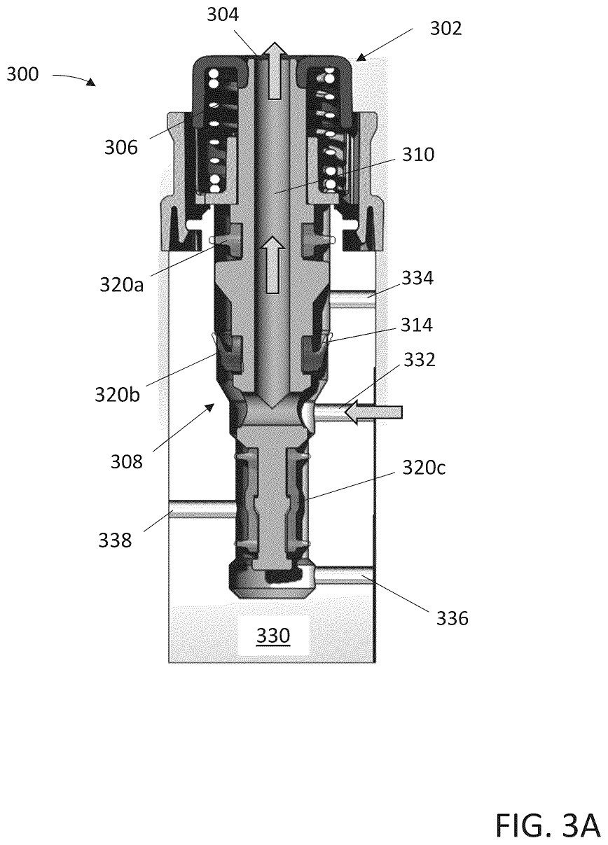

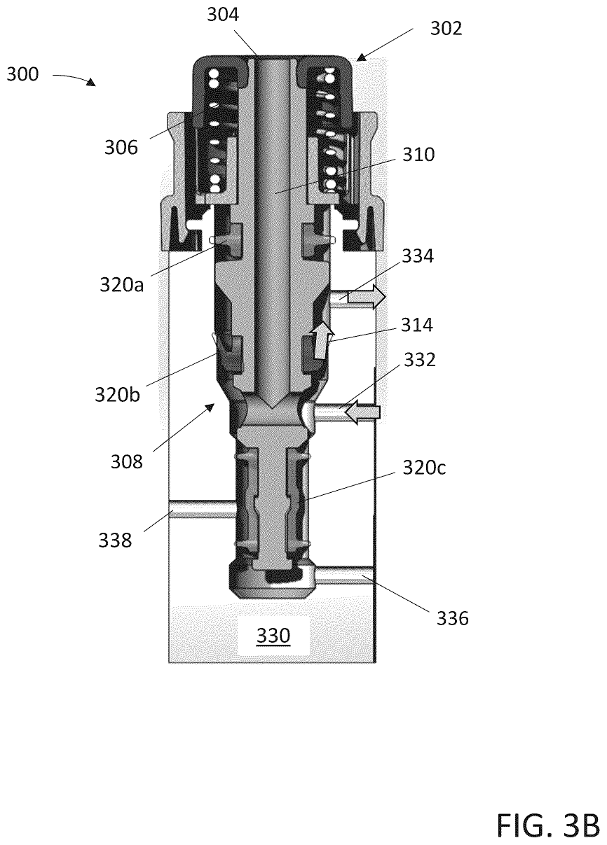

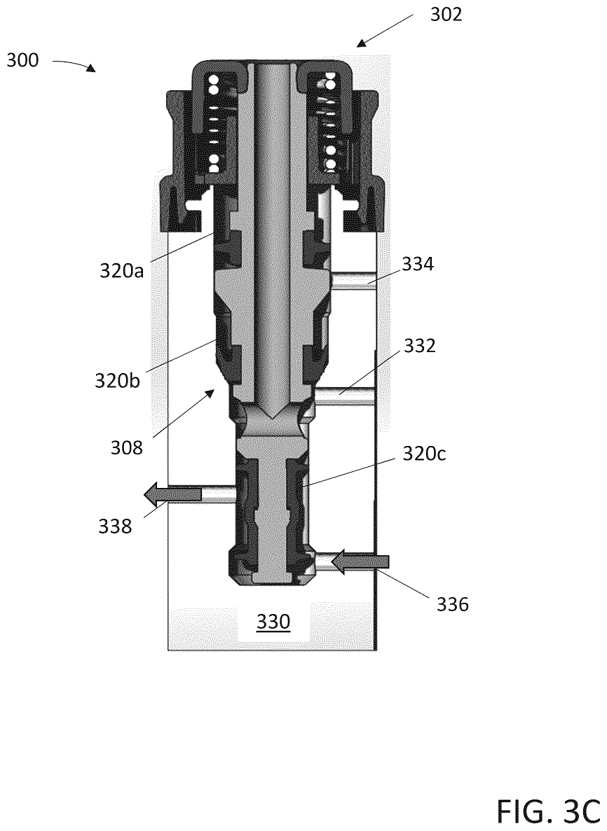

An example of a removable gas/water valve 300 is illustrated in FIG. 3 and in FIGS. 3A-3C. The valve cap 302 includes an air escape hole 304 and a spring member 306. The valve stem 308 includes a central lumen 310 connected to the air escape hole 304 and an air inlet 312.

The valve 300 is inserted into a body 330, such as one of the locations 135 described above and illustrated in FIGS. 1 and 2. The body 330 is sized and shaped to receive the stem 308 of the valve 300, as well as alternative valve designs (including each of those illustrated and described below). The valve body 330 includes an air inlet passage 332 communicating with a source of air as described above with respect to gas supply line 240a. An air outlet passage 334 similarly communicates with a gas feed line 240b.

FIG. 3A shows an open configuration for the valve when the air escape hole 304 is unblocked, air passing through the air inlet 312, up the central lumen 310, and out into the room. FIG. 3B shows a second configuration for the valve in which the air escape hole 304 is obstructed. In some implementations, the user may place a finger over the hole 304. A flap or other device may also be included for placement over the air escape hole 304 in other embodiments. When the air escape hole 304 is obstructed, air instead flows through a path defined by an exterior recess 314 in the valve stem 308 and the internal side wall of the body 330. Air passes through the air inlet passage 332, through the air outlet passage 334, and into the endoscope for use in insufflation as described.

Three seals 320a-c surround the valve stem 308 along its length, each including one or two wiper flanges configured to obstruct fluid flow when stationary without impeding vertical movement of the valve 300 within the body 330. An upper seal 320a is disposed below the valve cap 302 and above the exterior recess 314, obstructing flow in the valve well above the location of the air outlet passage 334. A middle seal 320b intersects the exterior recess 314 in the valve stem 308 but does not impede air flow when the valve 300 is in the upper position shown in FIGS. 3A and 3B. A lower seal 320c includes two wiper flanges.

The valve body 330 further includes a water inlet passage 336 connected to a water supply, and a water outlet passage 338 connected to a water feed line, at the lowest portion of the valve well. When the valve 300 is in the upper position as in FIGS. 3A and 3B, the lower of the two wiper flanges of the lower seal 320c sits above the water inlet passage 336, obstructing water from proceeding up the valve well or into the water outlet passage 338.

FIG. 3C shows a third configuration in which the valve 300 is positioned lower in the body 330. Downward force on the valve cap 302 causes and maintains this position; when the valve cap 302 is released, the spring member 306 returns the valve 300 to its previous position. In this configuration, the exterior recess 314 is no longer aligned with the air inlet 332 and air outlet 334 in the valve body 330. The middle seal 320b is seated along the inner wall of the body 330 to obstruct air flow above the air inlet 334. The two wiper flanges of the lower seal 320c are, in this configuration, positioned above the water outlet passage 338 and below the water inlet passage 336, creating an annular passage between the valve stem 308 and valve body 330 in which water can flow from the water inlet passage 336 to the water outlet passage 338. Upon release of the downward force and return of the valve cap 302 to its previous position, the placement of the lower seal 320c again prevents additional water from entering the feed through the annular passage.

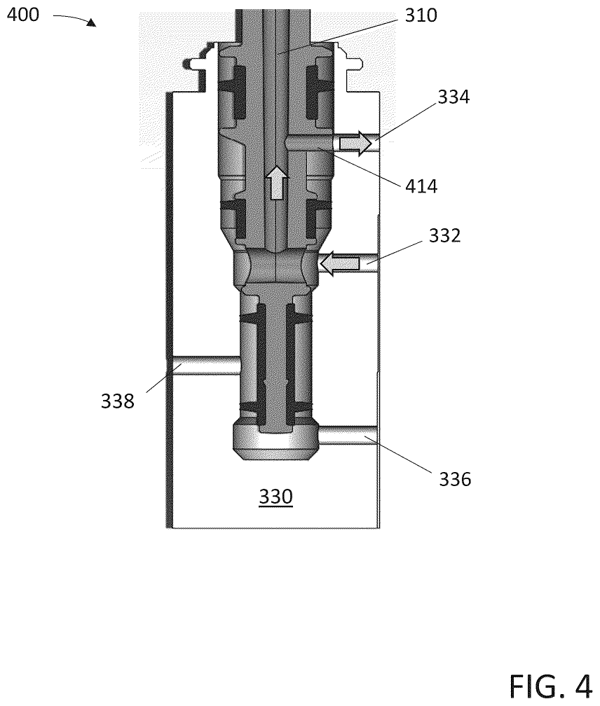

FIG. 4 shows a valve 400 with features similar to the valve 300 but with a notable change: an air outlet lumen 414 that connects to the central lumen 310. When the air escape hole 304 is obstructed, as in the second configuration shown in FIG. 3B, rather than air being diverted into an exterior recess, it instead flows through the air inlet 312, up through the central lumen 310, and out the air outlet lumen 414 to enter the air outlet passage 334. Because the diversion point of the air flow is farther downstream, the addition of the outlet lumen 414 may decrease the delay between obstructing the air escape hole 304 and insufflation. Furthermore, the internal lumen 414 may have a larger cross-section transverse to flow than the exterior recess, thus allowing for greater flow volume without impermissibly increasing air pressure.

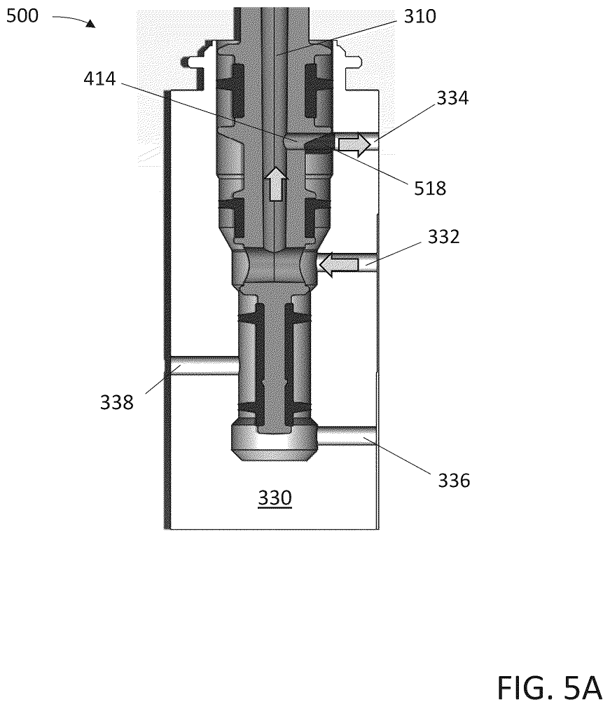

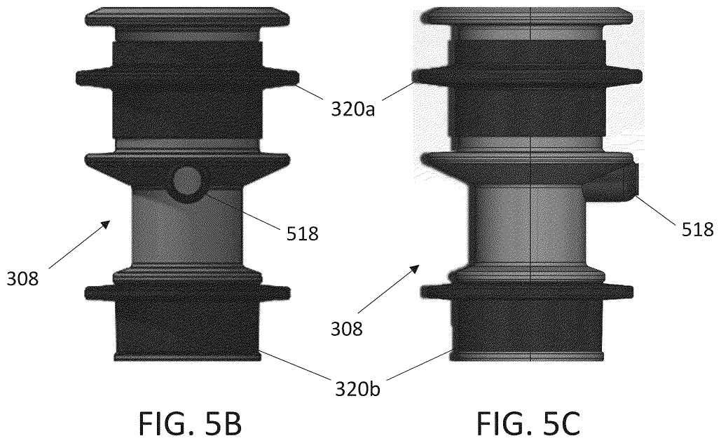

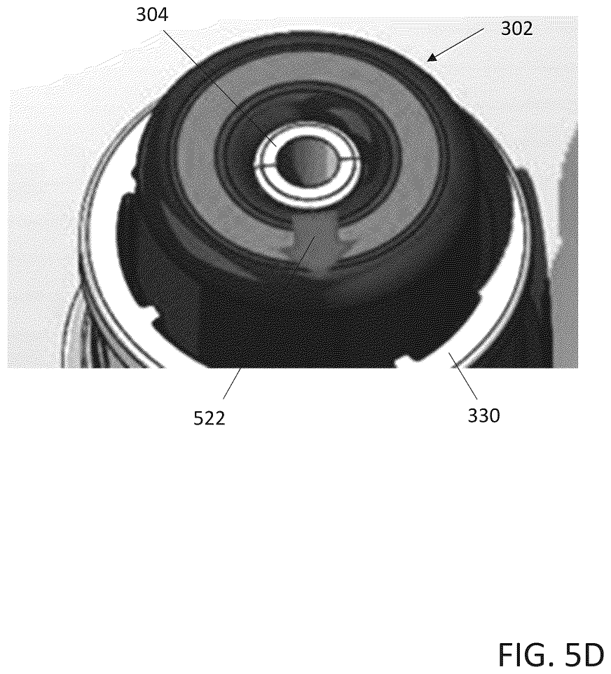

FIGS. 5A-D show a valve 500 with the altered air outlet path 414, but with the further addition of a seal 518 inserted within the air outlet path 414 for more secure interface with the valve well passage and connected air feed line. The seal 518 can be manufactured of a resilient material, and the outer lip can be shaped as a wiper seal to allow movement of the valve 500 up and down the valve well. As shown in FIG. 5D, an indicator 522 may be added to the valve cap 302 so that the valve 500 is placed in the well of the valve body 330 and the air outlet lumen 414 with the seal 518 is aligned with the air outlet passage 332 and air feed.

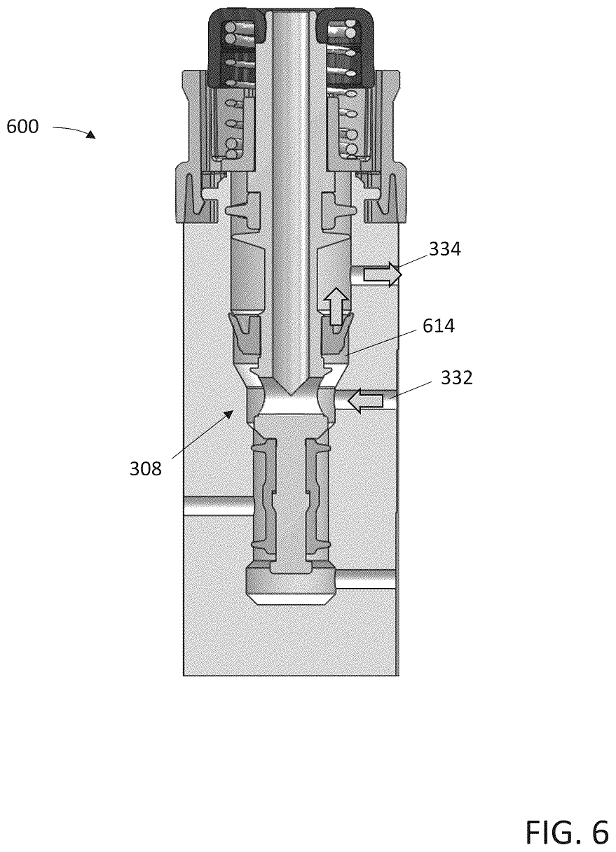

FIG. 6 shows a valve 600 with similar features to those of the valve 300 described above, but with a thinner profile corresponding to an increased exterior recess 614, providing a wider passage for air to travel between the air inlet 332 and air outlet 334. In some implementations, this recess reflects a change in cross-sectional diameter at all orientations of the stem 308 to preserve rotational symmetry. For example, where the inner wall of the valve body 330 has a diameter of 9.5 mm and the stem of the valve 300 as described above has a diameter at this same axial position of 6.8 mm, this valve 600 instead has a diameter of 3 mm at this same point. Thus, the recess 614 has a width of 3.25 mm per side, allowing for significantly greater air flow during insufflation.

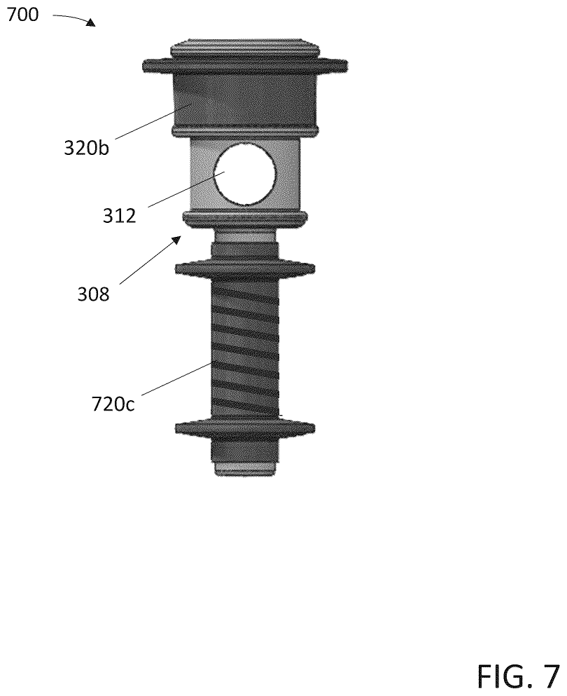

FIG. 7 shows a valve 700 with similar features to those of the valve 300 described above, but with a lower seal 720c having a ribbed surface for better directing the flow of water. Water entering the annular passage from the water inlet 336 is influenced by the shape of the seal 720c to flow in a particular direction, such as counter-clockwise, around the central portion of the valve stem 308 to exit via the water outlet 338. Other shapes and sizes for the textured features of the seal are possible as needed for the water feed as described above.

Although described as separate valve designs, it will be understood that the modifications to the seal 720c shown in FIG. 7 could be incorporated into any of the valves 400, 500, or 600 described with respect to FIGS. 4-6. Neither are the modifications restricted to the valve 300 herein described; one of ordinary skill will recognize that other valves may be modified in accordance with the improvements described herein.

The valve stem 308 may couple to the cap 302 in any suitable manner. In some cases, a portion (e.g., a proximal portion) of the valve stem 308 extending proximally of the air hole 304 may be coupled to the cap 302 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms.

The valve stem 308 may have any suitable configuration configured to adjust positions within the valve well, adjust flow paths to the air and water supplies and feeds, and couple to the cap 302.

The valve 300, 400, 500, 600, or 700 may be formed in any suitable manner. In some cases, though not required, the valve 300, 400, 500, 600, or 700 may be formed using a molding process, an injection molding process, a casting process, a finishing process, sanding, and/or by or with one or more additional or alternative manufacturing techniques. In one illustrative example, the valve 300, 400, 500, 600, or 700 may be formed using an injection molding process.

The valve stem 308 and the seals 320a-c and 518 may be formed from any suitable materials. In some cases, the valve stem 308 may be formed from a first material and the seals 320a-c and 518 may be formed from a second material, where the second material may be the same as or different than the first material. The valve stem 308 may be formed from a hard or rigid polymer and the seals 320a-c and 518 may be formed from a flexible polymer, but this is not required. In one example, the valve stem 308 may be formed from one or more of metals, polymer, acrylonitrile butadiene styrene (ABS), polycarbonate, and/or other suitable materials. In another example, the seals 320a-c and 518 may be formed from one or more of a polymer, thermoplastic elastomers (TPE), thermoplastic polyurethane (TPU), liquid silicone rubber (LSR), and/or other suitable materials.

The material of the seals 320a-c and 518 may have any suitable durometer. In one example, the material of the seals 320a-c and 518 when formed on the valve stem 308 may have a durometer in a range of about 20-80 shore A, about 30-60 shore A, and/or other suitable values within one or more other suitable ranges of durometer, but could be softer or firmer depending on the geometry used for the seals and the amount of interference desired with the inner wall of the valve body 330. In one example, the seals 320a-c and 518 may be formed from silicone with a durometer in a range of 40-50 shore A, but this is not required.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve assembly for a medical device, comprising:
a valve body having an air inlet passage, an air outlet passage, a water inlet passage, and a water outlet passage;
a valve cap having an air hole; and
a valve stem connected to the valve cap and configured to translate within the valve body between an upper position and a lower position, the valve stem comprising:
an air inlet,
a central lumen extending from the air inlet in the valve stem to the air hole in the valve cap, and
an air outlet lumen extending from the central lumen to a side wall of the valve stem, the air outlet lumen configured to fluidly communicate with the air outlet passage of the valve body when the valve stem is in the upper position.

2. The valve assembly of claim 1, further comprising:
an air outlet seal in the air outlet lumen, the seal configured to extend into the air outlet passage of the valve body when the valve stem is in the upper position and to move with the air outlet lumen without obstructing movement of the valve stem when the valve stem translates from the upper position to the lower position.

3. The valve assembly of claim 2, wherein the air outlet seal is a wiper seal.

4. The valve assembly of claim 2, wherein the air outlet seal is a flexible seal formed of a material with a durometer less than the durometer of a material forming the valve stem.

5. The valve assembly of claim 1, further comprising:
an upper seal extending circumferentially around the valve stem at an axial position above the air outlet lumen and below the valve cap, the upper seal having a wiper flange contacting the valve body when the valve stem is positioned within the valve body.

6. The valve assembly of claim 5, wherein the upper seal is a flexible seal formed of a material with a durometer less than the durometer of a material forming the valve stem.

7. The valve assembly of claim 1, further comprising:
a middle seal extending circumferentially around the valve stem at an axial position below the air outlet lumen and above the air inlet, the middle seal having a wiper flange contacting the valve body when the valve stem is in the lower position within the valve body.

8. The valve assembly of claim 7, wherein the middle seal is a flexible seal formed of a material with a durometer less than the durometer of a material forming the valve stem.

9. The valve assembly of claim 1, further comprising:
a lower seal extending circumferentially around the valve stem at an axial position below the air inlet, the lower seal having first and second wiper flanges positioned such that:
when the valve stem is in the upper position within the valve body, the second wiper flange is above the water inlet passage of the valve body and contacts the valve body to obstruct water flow between the water inlet passage and the water outlet passage; and
when the valve stem is in the lower position within the valve body, the first wiper flange is above the water inlet and outlet passages of the valve body and contacts the valve body to obstruct water flow from moving within the valve body above the lower seal, while the second wiper flange is below the water inlet and outlet passages of the valve body to permit water flow between the water inlet passage and the water outlet passage.

10. The valve assembly of claim 9, wherein the lower seal is a flexible seal formed of a material with a durometer less than the durometer of a material forming the valve stem.

11. The valve assembly of claim 9, the lower seal further comprising a seal body between the two wiper flanges having a ribbed surface configured to direct water flowing past the seal body in a preferred rotational direction.

12. A valve assembly for a medical device, comprising:
a valve body having an air inlet passage, an air outlet passage, a water inlet passage, and a water outlet passage;
a valve cap having an air hole; and
a valve stem connected to the valve cap and configured to translate within the valve body between an upper position and a lower position, the valve stem comprising:
an air inlet,
a central lumen extending from the air inlet in the valve stem to the air hole in the valve cap, and
a lower seal extending circumferentially around the valve stem at an axial position below the air inlet, the lower seal comprising:
a first wiper flange,
a second wiper flange below the first wiper flange, and
a seal body between the first and second wiper flanges comprising a ribbed surface configured to direct water flowing past the seal body in a preferred rotational direction;
wherein the lower seal is positioned on the valve stem such that:
when the valve stem is in the upper position within the valve body, the second wiper flange is above the water inlet passage of the valve body and contacts the valve body to obstruct water flow between the water inlet passage and the water outlet passage; and
when the valve stem is in the lower position within the valve body, the first wiper flange is above the water inlet and outlet passages of the valve body and contacts the valve body to obstruct water flow from moving within the valve body above the lower seal, while the second wiper flange is below the water inlet and outlet passages of the valve body to permit water flow between the water inlet passage and the water outlet passage.

13. The valve assembly of claim 12, wherein the lower seal is a flexible seal formed of a material with a durometer less than the durometer of a material forming the valve stem.

14. The valve assembly of claim 12, the valve stem further comprising an exterior recess such that, when the valve stem is in the upper position within the valve body, a passage is formed between the stem and the valve body to permit air flow between the air inlet passage and the air outlet passage.

15. The valve assembly of claim 12, the valve stem further comprising an air outlet lumen extending from the central lumen to a side wall of the valve stem, the air outlet lumen configured to fluidly communicate with the air outlet passage of the valve body when the valve stem is in the upper position.

16. The valve assembly of claim 15, further comprising:
an air outlet seal in the air outlet lumen, the seal configured to extend into the air outlet passage of the valve body when the valve stem is in the upper position and to move with the air outlet lumen without obstructing movement of the valve stem when the valve stem translates from the upper position to the lower position.

17. The valve assembly of claim 12, further comprising:
an upper seal extending circumferentially around the valve stem, the upper seal having a wiper flange contacting the valve body at an axial position below the valve cap and above the air outlet passage of the valve body when the valve stem is in the upper position within the valve body.

18. The valve assembly of claim 17, wherein the upper seal is a flexible seal formed of a material with a durometer less than the durometer of a material forming the valve stem.

19. The valve assembly claim 12, further comprising:
a middle seal extending circumferentially around the valve stem at an axial position above the air inlet, the middle seal having a wiper flange contacting the valve body when the valve stem is in the lower position within the valve body.

20. The valve assembly of claim 19, wherein the middle seal is a flexible seal formed of a material with a durometer less than the durometer of a material forming the valve stem.
